(19) United States
(12) Patent Application Publication
(10) Pub. No.: US 2024/0065673 A1
(43) Pub. Date: Feb. 29, 2024

(54) Ultrasound Smart Port Accessory

(71) Applicant: Bard Access Systems, Inc., Salt Lake City, UT (US)

(72) Inventors: Steffan Sowards, Salt Lake City, UT (US); William Robert McLaughlin, Bountiful, UT (US); Anthony K. Misener, Bountiful, UT (US)

(21) Appl. No.: 17/894,460

(22) Filed: Aug. 24, 2022

(51) Int. Cl. A61B 8/00; A61B 8/08; A61B 17/34

(52) U.S. Cl. CPC A61B 8/4411; A61B 8/085; A61B 8/4438; A61B 17/3403; A61B 2017/3413; A61B 2562/0219; A61B 2562/0223

(57) ABSTRACT

A medical ultrasound system that includes an ultrasound probe and an accessory interface apparatus configured to optionally couple with any medical device or system to enhance the functionality of the ultrasound system. The accessory apparatus exchanges data with the probe and receives power from the probe. The accessory apparatus includes processors and logic that governs its operation thereby adding functionality to the probe. The accessory apparatus receives input and provides output via any of a number of communication mechanisms, i.e., wireless, electrical, optical, inductive, RFID, IR, and the like. The optional accessories may add functionality to the probe, such as fiber optic shape sensing, obtaining electrical signals, obtaining bio-impedance measurements, tracking a needle, and determining the orientation of the probe. Additional accessories may include a needle guide, a triphalangeal support structure, or an acoustically transparent cap.

### BACKGROUND

Obtaining ultrasound images may be employed during a various medical procedures. Ultrasound systems and the associated ultrasound probes may be combined with a wide variety other medical devices and systems to enhance the performance of the medical procedures and reduce patient risk. However, the modification of the ultrasound systems or probes to accommodate integrated use with the other medical devices can be costly and logically complex. Systems and devices disclosed herein address the forgoing.

### SUMMARY

Briefly summarized, disclosed herein is an ultrasound system that includes a medical ultrasound system, having an ultrasound probe and an accessory interface apparatus coupled with the ultrasound probe. The accessory interface apparatus is configured to facilitate operational coupling between the ultrasound probe and one or more optional accessories, where the accessory interface apparatus includes a console having one or more processors and a non-transitory computer-readable storage medium having logic stored thereon that when executed by the one or more processors performs operations of the accessory interface apparatus that include (i) receiving input from one or more of the optional accessories via an input/output (I/O) portal of the accessory interface apparatus, and (ii) exchanging data between the accessory interface apparatus and the ultrasound probe.

In some embodiments, the system further includes a display coupled with the ultrasound probe, and the operations further include depicting information on the display pertaining to the operation of the one or more optional accessories.

In some embodiments, the ultrasound probe supplies electrical power to the accessory interface apparatus via at least one of electrical contacts or a magnetic field configured to inductively transfer the electrical power to the accessory interface apparatus.

In some embodiments, the accessory interface apparatus is configured to selectively attach to and detach from the ultrasound probe.

In some embodiments, the accessory interface apparatus is coupled with the ultrasound probe across a medical procedural barrier.

In some embodiments, the system further includes the medical procedural barrier and the medical procedural barrier includes a sheath covering the ultrasound probe.

In some embodiments, exchanging data takes place across at least one of a wireless connection or an optical connection.

In some embodiments, the I/O portal includes one or more of an electrical connection, an optical connection, or a wireless connection between the accessory interface apparatus and the one or more optional accessories.

In some embodiments, the accessory interface apparatus provides power to the one or more optional accessories.

In some embodiments, the one or more optional accessories includes one or more of an operator interface including buttons, a joystick, or a scroll wheel; fingerprint scanner; microphone; an IR receiver; an RFID reader; a scanner; or a camera.

In some embodiments, the accessory interface apparatus is configured to provide output to a clinician via one or more of a number of light indicators; a light projector; an audio transducer; or a haptic transducer.

In some embodiments, the accessory interface apparatus includes one or more of a needle guide connector configured for attachment of a needle guide thereto, a triphalangeal support structure, or an acoustically transparent cap.

In some embodiments, the input includes an RFID signal from at least one optional accessory indicating an identification of the at least one of the optional accessory, and the operations include configuring the accessory interface apparatus to receive and process input from the at least one optional accessory.

In some embodiments, the optional accessories include an elongate probe including an optical fiber having a number of sensors disposed along a length of the optical fiber. In such embodiments, the input includes optical signals, and the operations include processing the optical signals to determine one or more of (i) fluid or tissue motion adjacent the optical fiber, (ii) an image acquired by the optical fiber, (iii) a shape or strain of the optical fiber, or (iv) a motion of the optical fiber.

In some embodiments, the optional accessories include a magnetic tracking system configured to track a location and orientation of a needle and the input includes data pertaining to the location and orientation of the needle with respect to the ultrasound probe.

In some embodiments, the optional accessories include an elongate probe having a number of electrodes disposed along a length of the elongate probe. In such embodiments, the input includes electrical signals, and the operations include processing the electrical signals to determine one or more of a bio-impedance of a substance adjacent the elongate probe or an ECG waveform.

In some embodiments, the optional accessories include a blood vessel identification system, the input includes image data related to the identification and location of at least one blood vessel beneath the skin of a patient, and the operations include portraying an image of the blood vessel on the display in combination with the ultrasound image.

Also disclosed herein is an accessory interface apparatus for an ultrasound probe that includes (i) a probe interface configured to facilitate data and power exchange between the ultrasound probe and the accessory interface apparatus, (ii) an input/output (I/O) portal configured for receiving input from one or more optional accessories, and (iii) a console having one or more processors and a non-transitory computer-readable storage medium having logic stored thereon that when executed by the one or more processors performs operations of the accessory interface apparatus that include receiving the input from the one or more of the optional accessories and exchanging data between the accessory interface apparatus and the ultrasound probe, where the data pertains to the input.

In some embodiments, the input includes at least one of (i) optical signals that include reflected light having different spectral widths, or (ii) electrical signals that include an impedance measurements or an ECG signal.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is an illustration of an ultrasound imaging system including an accessory interface apparatus, in accordance with some embodiments;

FIG. 2 is a block diagram of a console of the accessory interface apparatus of FIG. 1, in accordance with some embodiments;

FIG. 3A is an illustration of an accessory including an electrical signal monitoring system coupled with the accessory interface apparatus, in accordance with some embodiments;

FIG. 3B is an illustration of an accessory including a fiber optic system coupled with the accessory interface apparatus, in accordance with some embodiments;

FIG. 3C is an illustration of an accessory including a bio-impedance system coupled with the accessory interface apparatus, in accordance with some embodiments;

FIG. 3D is an illustration of an accessory including a probe orientation monitoring system coupled with the accessory interface apparatus, in accordance with some embodiments;

FIG. 3E is an illustration of an accessory including a needle tracking system coupled with the accessory interface apparatus, in accordance with some embodiments;

FIG. 3F is an illustration of an accessory including a blood vessel identification device coupled with the accessory interface apparatus, in accordance with some embodiments;

FIG. 4A is an illustration of an accessory including a needle guide coupled with the accessory interface apparatus, in accordance with some embodiments;

FIG. 4B is an illustration of an accessory including a triphalangeal support structure coupled with the accessory interface apparatus, in accordance with some embodiments;

FIG. 4C is an illustration of an accessory including an acoustically transparent cap coupled with the accessory interface apparatus, in accordance with some embodiments; and

FIG. 5 is illustration of another embodiment of an ultrasound imaging system including an accessory interface apparatus, in accordance with some embodiments.

### DETAILED DESCRIPTION

Reference will now be made to figures wherein like structures will be provided with like reference designations. It is understood that the drawings are diagrammatic and schematic representations of exemplary embodiments of the present invention, and are neither limiting nor necessarily drawn to scale.

The phrases “connected to,” or “coupled with,” and “in communication with” refer to any form of interaction between two or more entities, including but not limited to mechanical, electrical, magnetic, electromagnetic, fluid, wireless, and optical interaction. Two components may be physically coupled with each other even though they are not in direct contact with each other. For example, two components may be physically coupled with each other through an intermediate component. Also, the words “including,” “has,” and “having,” as used herein, including the claims, shall have the same meaning as the word “comprising.”

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

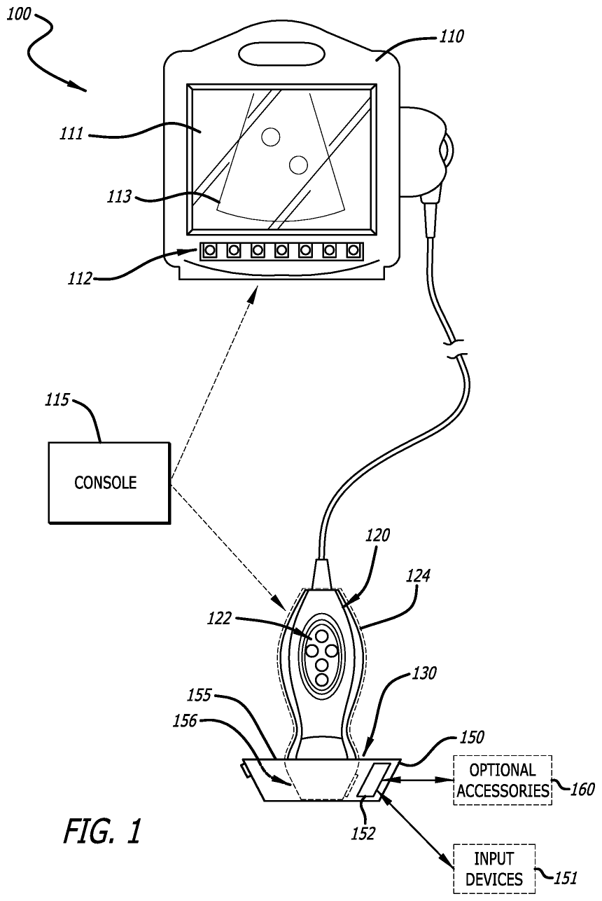

FIG. 1 illustrates an ultrasound imaging system (system) 100. The system 100 is generally configured for ultrasonically imaging a target area of a patient. The system 100 may be configured to facilitate the placement of a medical device (e.g., a guidewire, a catheter, a needle, or the like) within a patient vasculature. In some embodiments, the system 100 may be configured to facilitate defining a vascular access pathway for the medical device, such as via the insertion of a needle, for example. In some embodiments, the system 100 may further facilitate the positioning and/or orienting of the needle so that the needle may be accurately inserted into a target blood vessel.

The system 100 generally includes a display module 110, an ultrasound probe (probe) 120, and a console 115. The display module 110 includes a display 111. The display 111 and the probe 120 each include one or more user input controls 112, 122, respectively. The console 115 includes a number of console components (not shown) that govern the operation of the system 100. The console components may include inter alia one or more processors and memory (e.g., non-volatile memory or non-transitory, computer-readable storage medium) having logic stored thereon. Any portion of the console 115 may be included in the display module 110 and/or the probe 120. Briefly, the probe 120 is configured to (i) transmit ultrasonic signals from a head portion 121 thereof into a portion of a patient body and (ii) receive the ultrasonic signals after reflection by internal structures of the patient body. The system 100 processes the reflected ultrasonic signals for depiction on the display 111.

The system 100 further includes an accessory interface apparatus (apparatus) 150 coupled with the probe 120. The apparatus 150 includes a housing 155 configured to selectively attach to and detach from the probe 120. The apparatus 150 is a smart device, i.e., the apparatus 150 includes a console 156 disposed within the housing 155 as further described below.

The apparatus 150 is generally configured to facilitate operational coupling between the probe 120 and a number (e.g., 2, 3, 4, 5, 6 or more) of optional (accessories) 160 either one at a time or in combination. More specifically, the apparatus 150 provides for operative coupling between the probe 120 and the accessories 160. In some instances, an accessory 160 may be an active or smart device, and as such, operative coupling may include only the exchange of data. In other instances, an accessory 160 may be an active device that requires a supply of power to operate, and as such, operative coupling may include providing power to the accessory 160. In still other instances, an accessory 160 may be a passive device, such as an electrode or an optical fiber, for example. As such, operative coupling may include providing power and/or exchanging electrical or optical signals.

The apparatus 150 is configured to exchange data with the probe 120 via a probe interface 130. The probe interface 130 may include a number of a communication mechanisms either individually or in combination between the apparatus 150 and the probe 120. The communication mechanisms may include an electrical connection, a fiber optical coupling, an inductive (or magnetic field) coupling, or a wireless connection. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like. The data may include any information related to the operation of the apparatus 150. The data may also include identification data of the apparatus 150, such as a name, a model, a serial number, and/or manufacturing date, for example.

The apparatus 150 may receive electrical power from the probe 120 via the probe interface 130. In some embodiments, the apparatus 150 may optionally include a power source (e.g., battery) as an alternative to or to augment the electrical power received from the probe 120.

In some embodiments, the apparatus 150 may be coupled with the probe 120 across a medical procedural barrier 124. In some embodiments, the medical procedural barrier 124 includes a sheath that covers the probe 120. As such, the probe interface 130 may be configured to operate across the medical procedural barrier 124. In some embodiments, the system 100 may include the medical procedural barrier 124.

The apparatus 150 may be configured to obtain input data via one or more optional input devices 151. In some embodiments, the input devices 151 may include an operator interface having buttons, a joystick, or a scroll wheel for manually inputting data, or adjusting settings of the apparatus 150. In some embodiments, the input devices 151 may include a fingerprint scanner to obtain an identification of a clinician or the patient 50. In some embodiments, the input devices 151 may include a microphone to obtain and/or record audio information, where the audio information may include clinician or patient speech, audio output (e.g., alarms) of other medical equipment, or any other audible sounds during use of the system 100. In some embodiments, the input devices 151 may include an infrared (IR) receiver for receiving input via an IR or near IR connection. In some embodiments, the input devices 151 may include a camera.

The apparatus 150 includes an input/output (I/O) portal 152 configured to define operative coupling with (e.g., receiving input from and/or providing output to) the accessories 160 and/or the input devices 151. In some embodiments, the I/O portal may provide for communication with and/or power delivery to the accessories 160 and/or the input devices 151. The I/O portal 152 may include an electrical connection interface to enable to the apparatus 150 to electrically provide the output data and/or the power to the accessories 160. Alternatively, or in addition to the electrical connection interface, the output portal 132 may include an inductive (or magnetic field) interface to provide the output data and/or the power to the accessories 160. The output portal 152 may also include an optical interface to provide for light delivery to the accessories 160 and to provide for the exchange optical signals with the accessories 160. The output portal 152 may also include a wireless interface to facilitate wireless exchange of data with the accessories 160.

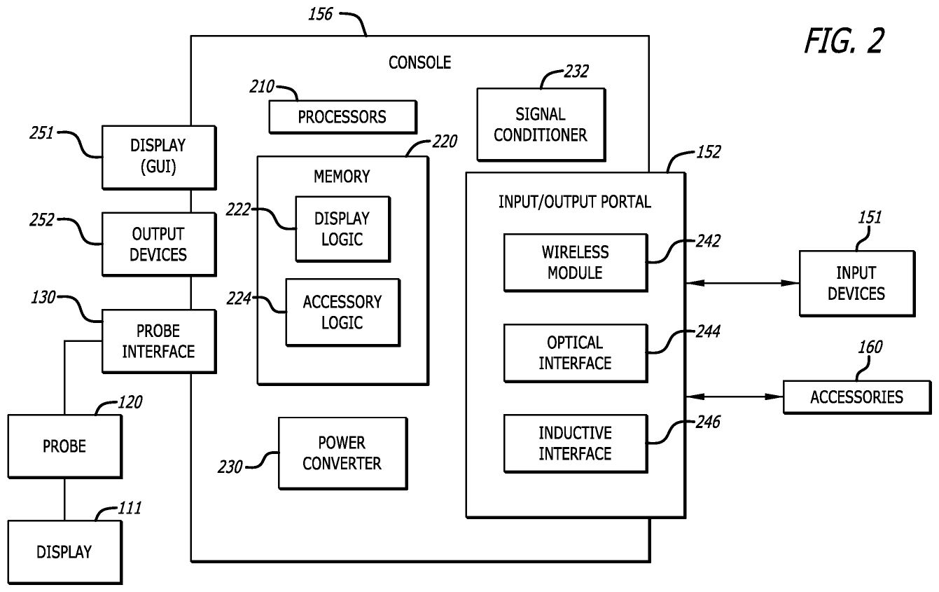

FIG. 2 illustrates a block diagram of a console of the apparatus. The apparatus 150 includes a console 156 configured to generally govern the operation of the apparatus 150, such as receiving input from the accessories 160 and/or the input devices and providing output to the display module 110 and/or other output devices, for example. The console 156 includes a number of processors 210 and memory 220 (e.g., non-transitory computer-readable medium) having logic stored thereon. For illustrative purposes, the logic may include display logic 222 and accessory logic 224. The console 156 further includes a power converter 230, a signal conditioner 232, and the I/O portal 152. Each of the components of the console 156 may be operatively coupled with each other to facilitate operation of the console 156. The console 156 is coupled with the input devices 151 and the accessories 160 via the I/O portal 152. The console 156 is coupled with the probe 120 and, in turn, the display module 111 via the probe interface 130.

In some embodiments, the console 156 (or the apparatus 150 generally) may include a user interface 251, such as a graphical user interface to enable to the clinician the operatively interact directly with the apparatus 150. For example, the clinician may turn the apparatus 150 “on” or “off”, access data within memory 220, change an operating mode, or enable a portion of the logic, via the user interface 251.

In some embodiments, the console 156 (or the apparatus 150 generally) may include a number of the output devices 252 such as a number of illuminating devices, an audio device, and or a haptic device. The output devices 252 may be configured to communicate an operating status of the apparatus 150, provide an alert, or otherwise provide notification to the clinician.

The console 156 receives power from the probe 120 via the probe interface 130. In some embodiments, the console 156 may include a battery in addition to or as an alternative to receiving power from the probe 120.

In some instances, the input may include an identity of the accessory 160. In such instances, the apparatus 150 may automatically transition to a mode of operation consistent with the identity of the accessory 160. By way of one example, the apparatus 150 may receive an RFID signal identifying the accessory 160 coupled with the apparatus 150 as an ECG electrode. In response to receiving the identification data, the apparatus 150 may automatically activate components and/or logic of the console 156 to facilitate obtaining an ECG signal from the ECG electrode and providing the ECG signal to the probe 120 and in turn to the display 111.

The I/O portal 152 may include one or more of a wireless module 242, an optical interface 244, or an inductive interface 246 to facilitate communication between the apparatus 150 and the input devices 151 and/or the accessories 160. As stated above, in some implementations, the accessories 160 may include standalone active medical devices/instruments. In some cases, the accessories 160 may include wireless capability. Such accessories 160 may be coupled with the apparatus 150 via the wireless module 242 or directly via a wired connection.

In some implementations, the accessories 160 may utilize optical fiber technology. Such accessories 160 may be coupled with the apparatus 150 via the optical interface 244. The optical interface 244 may include a light source (not shown) configured to provide light to an optical fiber of the accessory 160 and an optical receiver (also not shown) configured to receive optical signals from the optical fiber of the accessory 160 and convert the optical signals to electrical signals. In some embodiments, the optical interface 246 may enable optical coupling between the apparatus and the accessory 160 across the medical procedure barrier 124.

In some implementations, the accessories 160 may be configured to receive power from and/or exchange electrical signals with the apparatus 160 via the induction interface 246. In some embodiments, the induction interface 246 may enable power delivery and electrical signal exchange across the medical procedure barrier 124.

The signal conditioner 232 may be configured to receive raw data (e.g., electrical signals) from the I/O portal 152 and convert the raw data into digital data for processing by the processors 210. In some embodiments, the signal conditioner 232 may include an analog to digital converter and/or one or more operational amplifiers.

The display logic 222 may be generally configured to process input from the accessories 160 for portraying on the display 111. In some implementations, processing by the display logic 222 may include generating images (e.g., live images) of a waveform or other graphical presentation. In some implementations, processing by the display logic 222 may include portraying images of portions of an exterior or interior of the patient. For example, the accessory 160 may include a scope (e.g., a ureter scope) and the image may include an image of an anatomical element are other object within the patient.

The accessory logic 224 may be generally configured to enable operation of the accessories 160. As stated above multiple accessories 160 may be operatively coupled with probe 120 at the same time. As such, the accessory logic 224 may enable operation of a combination of multiple accessories 160. The accessory logic 224 may be configured to enable combined operation of the probe 120 and the accessories 160.

In some embodiments, all or a portion of the logic for a given accessory 160 may be stored in the memory 220. For example, in some implementations, the accessory 160 may include memory having accessory operational logic stored thereon, and upon coupling of the accessory 160, the accessory operational logic may be processed by the processors 210 of the console 156. In some implementations, the accessory operational logic may be transferred to and stored in the memory 220.

The apparatus 150 may be configured to optionally provide output via the output devices 252. For example, the apparatus 150 may provide an event notification via the audio device or the haptic device for various events during a procedure, such as a cannulation of the target blood vessel, a vein/artery confirmation, or a deviation of a needle from vasculature access pathway, for example. Similarly, as the probe 120 is hand-held, the apparatus 150 may cause a vibration of the probe 120 via the haptic device in response to various procedural events. In some embodiments, the apparatus 150 may generate a data packet of procedural events and send the data packet to the probe 120 via the probe interface 130.

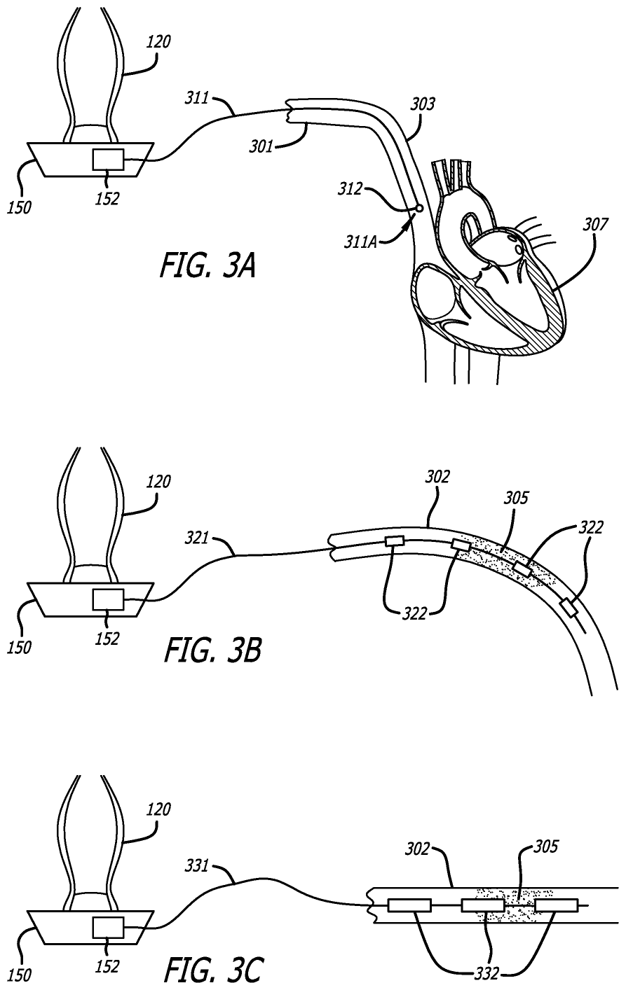

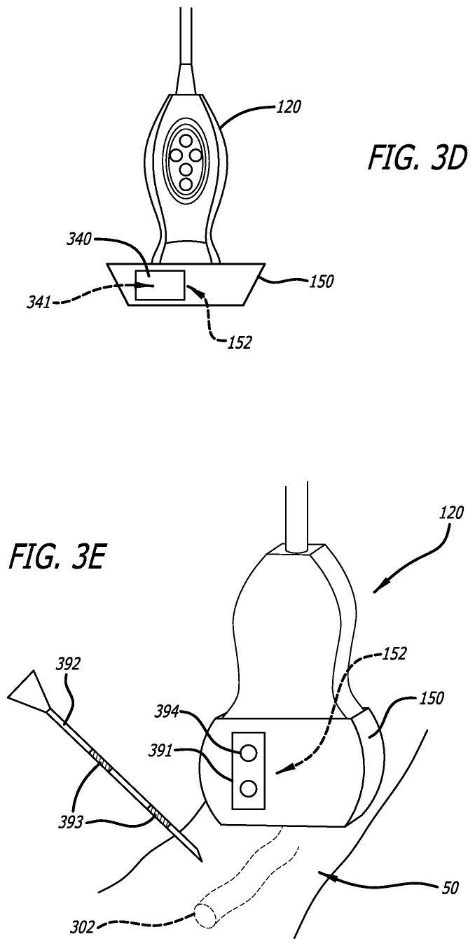

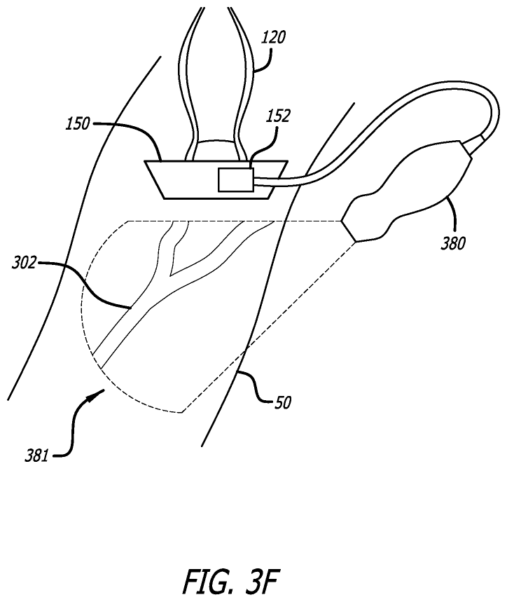

FIGS. 3A-3F illustrate various implementations of the apparatus 150, i.e., various optional accessories 160 that may be operatively coupled with the apparatus 150. FIG. 3A illustrates an elongate medical device 311 configured for advancement along a vasculature 301 of the patient, such as the superior vena cava 303 entering the heart 307 of the patient, for example. The elongate medical device 311 includes an electrode 312 at a distal end 311A, where the electrode 312 is configured to obtain an electrical signal from the patient such as an electro-cardiogram (ECG) signal, for example. In such an implementation, the elongate medical device 311 may be coupled with the apparatus 150 via the I/O portal 152, i.e., an electrical connection of the I/O portal 152. The signal conditioner 232 may convert the raw ECG signal of the electrode 312A into digital data for processing by the processors 210. The accessory logic 224 may derive an ECG waveform from the digital data and the display logic 222 may portray the ECG waveform on the display 111. In some implementations, the display logic 222 may portray the ECG waveform on the display 111 in combination with an ultrasound image from the probe 120 and/or information pertaining to an additional accessory 160.

FIG. 3B illustrates a fiber optic accessory 320 that includes a multi-core optical fiber 321 having a number of sensors (e.g., Fiber Bragg Gratings) 322 disposed along the optical fiber 321. The optical fiber 321 may be configured for advancement along a blood vessel 302 of the patient 50 so that the fiber optic system 320 may acquire data related to the blood vessel 302. In some embodiments, the fiber optic system 320 may determine a fluid (e.g., blood 305) or tissue motion adjacent the optical fiber 321 via the doppler effect. The fiber optic system 320 may further acquire data related to a strain, a shape, or a motion of at least a portion of the optical fiber 321 when the optical fiber 321 is advanced along the blood vessel 302. The fiber optic system 320 may also obtain images via the optical fiber 321, such as an image of an interior of a blood vessel, for example. In such an implementation, the multi-core optical fiber 321 may be coupled with the apparatus 150 via the I/O portal 152, i.e., the optical interface 244. The optical interface 244 may provide a broadband incident light and/or a coherent light to the multi-core optical fiber 321 and receive optical signals from the multi-core optical fiber 321. The optical interface 244 may convert the optical signals into raw electrical signals. The signal conditioner 232 may convert the raw electrical signals into digital data for processing by the processors 210. The accessory logic 224 may derive, from the raw electrical signals, one or more of a blood flow, shape or strain of the optical fiber 321, a motion of the optical fiber 321, and/or an image obtained by the optical fiber 321, and the display logic 222 may portray information and/or images pertaining thereto on the display 111. In some implementations, the display logic 222 may portray the information and/or images on the display 111 in combination with an ultrasound image from the probe 120 and/or information pertaining to an additional accessory 160.

FIG. 3C illustrates an accessory that includes an elongate medical device 331 (e.g., a catheter, a guidewire, a needle, or a stylet) that may be inserted into the patient, such as advancement along a blood vessel 302, for example. The elongate medical device 331 includes two or more electrodes 332 distributed along the elongate medical device 331. The two or more electrodes 332 are configured to obtain an electrical impedance of a bodily substance, such as the blood 305 within the blood vessel 302, for example. In some embodiments, the elongate medical device 331 may be configured for insertion into body tissue so that the two or more electrodes 332 may obtain an electrical bio-impedance of the tissue. In such an implementation, the elongate medical device 331 may be coupled with the apparatus 150 via the I/O portal 152, i.e., an electrical connection of the I/O portal 152. The signal conditioner 232 may convert the raw electrical impedance signal of the elongate medical device 331 into digital data for processing by the processors 210. The accessory logic 224 may derive an impedance measurement from the digital data and the display logic 222 may portray the impedance measurement on the display 111. In some implementations, the display logic 222 may portray the impedance measurement on the display 111 in combination with an ultrasound image from the probe 120 and/or information pertaining to an additional accessory 160.

FIG. 3D illustrates an accessory in the form of a probe orientation monitoring system 340. The probe orientation monitoring system 340 is attached to the apparatus 150 or the probe 120. The probe orientation monitoring system 340 includes an inertia measurement unit (IMU) 341 including a gyroscope and/or an accelerometer configured to determine an orientation and/or position of the probe 120. In some instances, the clinician may reposition the probe 120 during a procedure. As such, the probe orientation monitoring system 340 is configured to monitor and determine the orientation and/or position of the probe 120 during movement of the probe 120. In this exemplary implementation, the probe orientation monitoring system 340 includes one or processors that govern the operation of the IMU 341 according to logic of the probe orientation monitoring system 340. In some implementations, the probe orientation monitoring system 340 may transfer all or a portion of the logic of the probe orientation monitoring system 340 to the memory 220 for processing by the processors 210. In some implementations, the apparatus 150 may provide power to the probe orientation monitoring system 340 and exchange data with the probe orientation monitoring system 340 via a wired connection. In other embodiments, the probe orientation monitoring system 340 may be communicatively coupled with the apparatus 150 via a wireless connection. The accessory logic 224 may derive or receive probe orientation/position data from the probe orientation monitoring system 340 and the display logic 222 may portray the probe orientation/position information on the display 111. In some implementations, the display logic 222 may portray the probe orientation/position information on the display 111 in combination with an ultrasound image from the probe 120 and/or information pertaining to an additional accessory 160.

FIG. 3E illustrates an accessory that includes a needle tracking system 391 configured to track (e.g., magnetically track) the orientation and/or position of a needle 392 with respect to the probe 120 during use, such as during insertion of the needle 392 within a blood vessel 302 of the patient 50. In some embodiments, the needle 392 may include a number of magnetic elements 393 and the needle tracking system 391 may include a number of magnetic sensors 394. In some implementations, the needle tracking system 391 includes a number of magnetic sensors 394 that are physically attached to the apparatus 150 or the probe 120. The magnetic sensors 394 receive power from the apparatus 150 and provide raw electrical signals to the apparatus 150. In such an implementation, the elongate medical device 311 may be coupled with the apparatus 150 via the I/O portal 152, i.e., an electrical connection of the I/O portal 152. The signal conditioner 232 may convert the raw electrical signals into digital data for processing by the processors 210. The accessory logic 224 may derive the position and/or orientation of the needle 392 and the display logic 222 may portray an image of the needle 392 in relation to the probe 120 on the display 111. In some implementations, the display logic 222 may portray the image of the needle 392 on the display 111 in combination with an ultrasound image from the probe 120 and/or information pertaining to an additional accessory 160.

FIG. 3F illustrates an accessory that includes a blood vessel identification device 380 configured to locate and identify blood vessels 302 beneath the skin of the patient 50. The blood vessel identification device 380 utilize infrared scanning to locate and identify blood vessels 302. In some embodiments, the blood vessel identification device 380 may be configured to project an image 381 of the blood vessels 302 on the skin surface of the patient so that the clinician visualize the blood vessels 302 that are located beneath the image. In such an implementation, the blood vessel identification device 380 may be coupled with the apparatus 150 via the I/O portal 152, i.e., an electrical connection of the I/O portal 152. The accessory logic 224 may receive the image data from the vessel identification device 380 and the display logic 222 may portray an image 381 (or similar image) in relation to the probe 120 on the display 111. In some implementations, the display logic 222 may portray the image 381 on the display 111 in combination with an ultrasound image from the probe 120 and/or information pertaining to an additional accessory 160.

The exemplary optional accessories 160 described above in relation to FIGS. 3A-3F are mere examples of many different types of accessories that may be coupled with the apparatus 150 as may be appreciated by one of ordinary skill. By way of summary, the apparatus 150 may be configured to couple with any suitable medical device, or medical system that may add functionality to the probe 160.

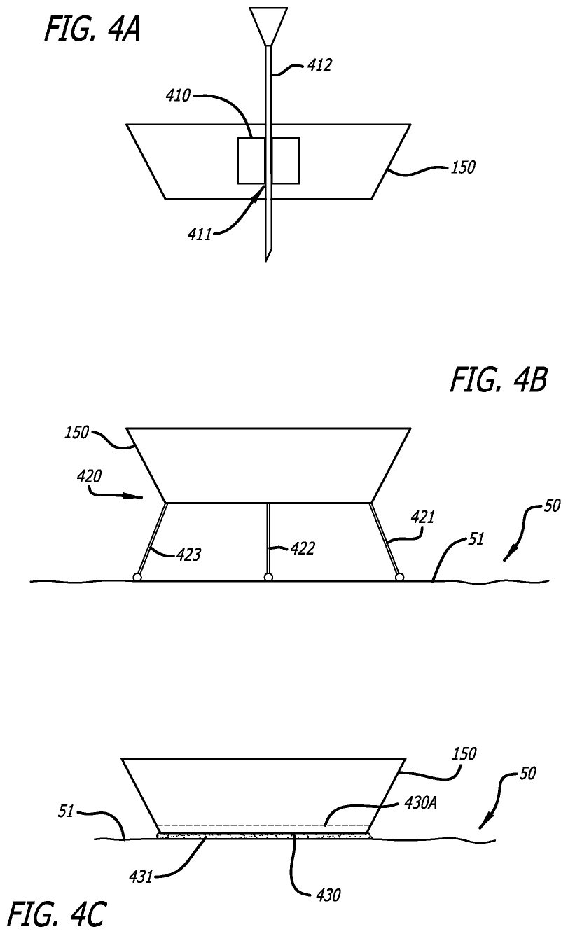

FIGS. 4A-4C illustrate additional accessories that may be coupled with or integrated into the apparatus 150 in additional to the accessories 160.

FIG. 4A illustrates the apparatus 150 including a needle guide 410 attached thereto. The needle guide 410 includes a channel 411 configured for receiving a needle 412 therein such that the needle guide 410 defines a lateral position and an orientation of the needle 412 with respect to the accessory 450 (i.e., the probe 120) during use. In some embodiments, the apparatus 150 may alternatively include a needle guide connector (not shown) configured for selective attachment to and detachment from the needle guide 410.

FIG. 4B illustrates the apparatus 150 including a triphalangeal stabilization structure 420 (e.g., a tripod structure) having three support members (legs) 421-423 extending between the apparatus 150 and the skin surface 51 of the patient 50. The triphalangeal stabilization structure 420 is configured to enhance the stabilization of the probe 120 when the probe 120 is placed on the patient.

FIG. 4C illustrates the apparatus 150 including a lens cap 430 configured for disposition between the probe 120 and the skin surface 51, where the lens portion 430A of the lens cap 430 is ultrasonically transparent. In some embodiments, the lens cap 430 includes a gel 431 to enhance an ultrasonic coupling of the probe 120 with the skin surface 51.

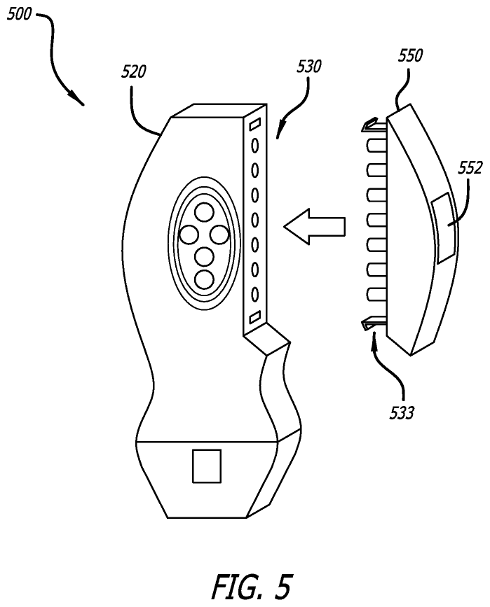

FIG. 5 illustrates another embodiment of an ultrasound imaging system 500 that can, in certain respects, resemble the components and functionality of the ultrasound imaging system 100 described in connection with FIGS. 1-3F. It will be appreciated that all the illustrated embodiments may have analogous features. Accordingly, like features are designated with like reference numerals with a leading digit of “5.” Relevant disclosure set forth above regarding similarly identified features thus may not be repeated hereafter. Moreover, specific features of the ultrasound imaging system and related components shown in FIGS. 1-3F may not be shown or identified by a reference numeral in the drawing or specifically discussed in the written description that follows. However, such features may clearly be the same, or substantially the same, as features depicted in other embodiments and/or described with respect to such embodiments. Accordingly, the relevant descriptions of such features apply equally to the features of the ultrasound imaging system of FIG. 5. Any suitable combination of the features, and variations of the same, described with respect to the ultrasound imaging system and components illustrated in FIGS. 1-3F can be employed with the ultrasound imaging system and components of FIG. 5, and vice versa.

The ultrasound imaging system (system) 500 generally includes the probe 520 and the accessory interface apparatus (apparatus) 550. The system 500 differs from the system 100 of FIGS. 1-3F in that the apparatus 550 couples with the probe 520 along a side of the probe 520 versus at the head of the probe. Similar to the system 100, the system 500 includes a probe interface 530 that may in some respects resemble the components and functionality of the probe interface 130. Similar to the apparatus 150, the apparatus 550 includes an I/O portal 552 that may in some respects resemble the components and functionality of the I/O portal 152. In some embodiments, the probe interface 530 may include one or more attachment devices 533, such as clips, for example, to define a secure attachment of the apparatus 550 to the probe 520.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A medical ultrasound system, comprising:
an ultrasound probe; and
an accessory interface apparatus coupled with the ultrasound probe, the accessory interface apparatus configured to facilitate operational coupling between the ultrasound probe and one or more optional accessories,
wherein the accessory interface apparatus includes a console having one or more processors and a non-transitory computer-readable storage medium having logic stored thereon that when executed by the one or more processors performs operations of the accessory interface apparatus that include:
receiving input from one or more of the optional accessories via an input/output (I/O) portal of the accessory interface apparatus, and
exchanging data between the accessory interface apparatus and the ultrasound probe.

2. The system of claim 1, further comprising a display coupled with the ultrasound probe, the operations further including depicting information on the display pertaining to the operation of the one or more optional accessories in combination an ultrasound image obtained by the ultrasound probe.

3. The system of claim 2, wherein:
the optional accessories include a blood vessel identification system;
the input includes image data related to the identification and location of at least one blood vessel beneath the skin of a patient; and
the operations include portraying an image of the blood vessel on the display in combination with the ultrasound image.

4. The system of claim 1, wherein the accessory interface apparatus receives electrical power from the ultrasound probe via at least one of electrical contacts or a magnetic field configured to inductively transfer the electrical power to the accessory interface apparatus.

5. The system of claim 1, wherein the accessory interface apparatus is configured to selectively attach to and detach from the ultrasound probe.

6. The system of claim 1, wherein the accessory interface apparatus is coupled with the ultrasound probe across a medical procedural barrier.

7. The system of claim 6, further comprising the medical procedural barrier, the medical procedural barrier including a sheath covering the ultrasound probe.

8. The system of claim 1, wherein exchanging data takes place across at least one of a wireless connection or an optical connection.

9. The system of claim 1, wherein the I/O portal includes one or more of an electrical connection, an optical coupling, an inductive coupling or a wireless connection between the accessory interface apparatus and the one or more optional accessories.

10. The system of claim 1, wherein the accessory interface apparatus provides power to the one or more optional accessories.

11. The system of claim 1, further comprising input devices configured to provide input to the accessory interface apparatus via the I/O portal, wherein the input devices include one or more of:
an operator interface including buttons, a joystick, or a scroll wheel;
fingerprint scanner;
microphone;
an IR receiver;
an RFID reader;
a scanner; and
a camera.

12. The system of claim 1, wherein the accessory interface apparatus is configured to provide output to a clinician via one or more of:
a number of illuminating devices;
an audio device; and
a haptic device.

13. The system of claim 1, wherein the optional accessories include one or more of:
a needle guide connector configured for attachment of a needle guide thereto,
a triphalangeal support structure, and
an acoustically transparent cap.

14. The system of claim 1, wherein:
the input includes an RFID signal from at least one of the optional accessories indicating an identification of the at least one of the optional accessories, and
the operations include configuring the accessory interface apparatus to receive and process input from the at least one of the optional accessories.

15. The system of claim 1, wherein:
the optional accessories include an elongate probe including an optical fiber having a number of sensors disposed along a length of the optical fiber,
the input includes optical signals, and
the operations include processing the optical signals to determine one or more of:
fluid or tissue motion adjacent the optical fiber;
an image acquired by the optical fiber;
a shape or strain of the optical fiber; and
a motion of the optical fiber.

16. The system of claim 1, wherein:
the optional accessories include a magnetic tracking system configured to track a location and orientation of a needle, and
the input includes data pertaining to the location and orientation of the needle with respect to the ultrasound probe.

17. The system of claim 1, wherein:
the optional accessories include an elongate probe having a number of electrodes disposed along a length of the elongate probe,
the input includes electrical signals, and
the operations include processing the electrical signals to determine one or both of a bio-impedance of a substance adjacent the elongate probe and an ECG waveform.

18. An accessory interface apparatus for an ultrasound probe, comprising:
a probe interface configured to facilitate data and power exchange between the ultrasound probe and the accessory interface apparatus;
an input/output (I/O) portal configured for receiving input from one or more optional accessories; and
a console having one or more processors and a non-transitory computer-readable storage medium having logic stored thereon that when executed by the one or more processors performs operations of the accessory interface apparatus that include:
receiving the input from the one or more of the optional accessories, and
exchanging data between the accessory interface apparatus and the ultrasound probe, the data pertaining to the input.

19. The apparatus of claim 18, wherein the input includes one or both of optical signals that include reflected light having different spectral widths, and electrical signals that include an impedance measurement or an ECG signal.
